(19) United States
(12) Patent Application Publication
(10) Pub. No.: US 2026/0102182 A1
(43) Pub. Date: Apr. 16, 2026

(54) TIGHT STENOSIS LESION ROTATIONAL ATHERECTOMY DEVICE AND METHODS OF USE

(71) Applicant: Cardiovascular Systems, Inc., St. Paul, MN (US)

(72) Inventors: Devon R. Holley, Ramsey, MN (US); William W. Barbera, Minneapolis, MN (US); Grant A. Adams, New Brighton, MN (US)

(21) Appl. No.: 19/352,888

(22) Filed: Oct. 8, 2025

Related U.S. Application Data
(60) Provisional application No. 63/705,917, filed on Oct. 10, 2024.
(60) Provisional application No. 63/800,611, filed on May 6, 2025.

(51) Int. Cl. A61B 17/3207; A61B 17/32

(52) U.S. Cl. CPC A61B 17/320758; A61B 2017/320004; A61B 2017/320056

(57) ABSTRACT

A device for creating a pilot hole through lesions in body passageways including an elongated flexible drive shaft advanceable over a guide wire, the drive shaft having a proximal end and a distal end, and a piloting head fixedly attached to at least a portion of the distal end of the driveshaft, wherein the piloting head comprises a distal end having a first diameter, a proximal end having a second diameter that is larger than the first diameter, and an intermediate shoulder area comprising a third diameter that is larger than the first and second diameters, wherein the intermediate shoulder area comprises a radiused crest having a backside area having an abrasive coating, and wherein the diameter of the piloting head tapers down at a generally constant angle from the intermediate shoulder area to the proximal end of the piloting head.

### CROSS-REFERENCE TO RELATED APPLICATIONS

This application claims priority to and the benefit of U.S. Provisional Application No. 63,705,917, filed Oct. 10, 2024, and entitled CHRONIC TOTAL OCCLUSION LESION PILOTING DEVICE AND METHODS OF USE, and U.S. Provisional Application No. 63/800,611, filed May 6, 2025, and entitled CHRONIC TOTAL OCCLUSION LESION PILOTING DEVICE AND METHODS OF USE, the entire contents of which are incorporated herein by reference in their entireties.

### TECHNICAL FIELD

The present invention is directed generally to devices and methods of removing material from body passageways such as arteries, and more specifically is directed to atherectomy devices and methods for treating tight stenosis lesions by creating a pilot hole through the lesions, thereby enabling other devices to treat previously inaccessible treatment areas.

### BACKGROUND

A variety of techniques and instruments have been developed for use in the removal or repair of tissue in arteries and similar body passageways. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, with the blocking material being referred to as stenotic material. If left untreated, such stenoses can cause angina, hypertension, myocardial infarction, strokes and the like.

Rotational atherectomy procedures have become a common technique for removing such stenotic material. Such procedures are used most frequently to initiate the opening of calcified lesions in coronary arteries. Most often the rotational atherectomy procedure is not used alone, but is followed by a balloon angioplasty procedure, which, in turn, is very frequently followed by placement of a stent to assist in maintaining patency of the opened artery. For non-calcified lesions, balloon angioplasty most often is used alone to open the artery, and stents often are placed to maintain patency of the opened artery. Studies have shown, however, that a significant percentage of patients who have undergone balloon angioplasty and had a stent placed in an artery experience stent restenosis, which is a blockage of the stent that most frequently develops over a period of time as a result of excessive growth of scar tissue within the stent. In such situations an atherectomy procedure is the preferred procedure to remove the excessive scar tissue from the stent (balloon angioplasty being not very effective within the stent), thereby restoring the patency of the artery.

In some cases, the lesion may completely or almost completely occlude the artery, which is referred to as a Chronic Total Occlusion (CTO) situation in which physicians may only be able to cross the lesion using a standard guide wire. In these cases, the lesion inner lumen is so small that it prevents microcatheters, balloon catheters, and other medical devices from crossing the lesion, which also prevents treatment of the artery. These cases are difficult for physicians, as they often cannot recognize that the lesion is uncrossable until they unsuccessfully try to use multiple devices that are blocked from entering the artery. This trial-and-error process can lead to using multiple expensive medical devices and significantly prolonging the medical procedure.

Thus, there is a need to provide devices and methods to first debulk an uncrossable lesion to create a pilot hole before using other devices to further treat the artery. Such devices and methods are described herein that facilitate opening pilot holes through difficult occlusions, lesions, and/or stenosis.

### BRIEF DESCRIPTION OF THE DRAWINGS

The present invention will be further explained with reference to the appended Figures, wherein like structure is referred to by like numerals throughout the several views, and wherein:

FIG. 1 is a perspective view of a prior art rotational atherectomy device;

FIG. 2 is a block diagram of a typical rotational atherectomy device;

FIG. 3 is a perspective view of a rotational atherectomy device of the type shown in FIG. 1, and including a control unit;

FIG. 4 is a perspective view of an exemplary piloting head of the invention;

FIG. 5 is a front view of the exemplary piloting head illustrated in FIG. 4;

FIG. 6 is a graph that compares the time to cross a calcified lesion and the amount of wire abrasion with device spin speed;

FIG. 7 is a diagram that shows driveshaft variables with respect to the number of driveshaft wires or filars used;

FIG. 8 is a graph that illustrates relationships between the number of driveshaft wires or filars and the distance that the device travels or jumps upon crossing a lesion;

FIG. 9 is a front view of a rotational atherectomy device that includes a piloting head at its distal end;

FIG. 10 illustrates several exemplary piloting heads, in accordance with the invention;

FIGS. 11A-11D illustrate four sequential steps of using a rotational atherectomy device in a vessel to create a pilot hole in an uncrossable lesion before using other devices to treat the vessel;

FIG. 12 is a side view of an exemplary handle for use with an atherectomy device, in accordance with the invention;

FIGS. 13A and 13B are top views of two exemplary handles for use with an atherectomy device, wherein the handle of FIG. 13B has a shorter profile than the handle of FIG. 13A, and wherein FIG. 13B is a top view of the exemplary handle of FIG. 12;

FIG. 14 is a perspective view of a tip component of the handle of FIG. 12, further illustrating a portion that is insertable into an end of the handle;

FIG. 15 is a perspective view of the inner portion of the handle of FIG. 12;

FIGS. 16A and 16B are perspective views from different angles of an exemplary handle for use with an atherectomy device, in accordance with the invention;

FIG. 17 is a cross-sectional view of an exemplary piloting head for use with an atherectomy device, in accordance with the invention;

FIG. 18 is a cross-sectional view of another exemplary piloting head for use with an atherectomy device, in accordance with the invention;

FIG. 19 is a cross-sectional view of another exemplary piloting head for use with an atherectomy device, in accordance with the invention;

FIG. 20 is a perspective view of another exemplary piloting head for use with an atherectomy device, in accordance with the invention;

FIG. 21 is a perspective view of another exemplary piloting head for use with an atherectomy device, in accordance with the invention; and

FIG. 22 is a perspective view of another exemplary piloting head for use with an atherectomy device, in accordance with the invention.

### DETAILED DESCRIPTION

The devices and methods described herein are directed to an atherectomy device that utilizes a front-facing, diamond-coated tip to pilot through otherwise uncrossable lesions in arteries. The device includes a motor that spins the crown at a rotational speed (e.g., approximately 40 kRPM) that effectively drills away lesion material, creating a small pilot hole (e.g., approximately 1.0 mm diameter, although smaller or larger pilot hole sizes are contemplated) through the lesion to allow for subsequent treatment of the artery. Other abrasive coatings in addition to or instead of diamond coatings are also contemplated for the tip.

By utilizing an exchangeable handle platform, the devices and methods described herein generally include two main design features that support subsequent treatment. First, an exchangeable handle includes the ability to remove a deployed guide wire and backload a new guide wire through the device handle's strain relief. In this sense, the device can be utilized as a type of exchange catheter, streamlining the ability of physicians to place their desired guide wire for lesion treatment. Second, the device may utilize removable cartridges that include the device's driveshaft and diamond-coated crown. This system configuration allows for the CTO cartridge to be replaced with a subsequent treatment cartridge after piloting through the lesion, such as an orbital atherectomy cartridge or a larger sized rotational atherectomy cartridge. Another subsequent treatment can include the use of a balloon similar to an angioplasty balloon that is filled with a conductive liquid medium for expanding the balloon in position at the lesion or thrombus, and that includes one or more pairs of electrodes (emitters) operatively positioned within the conductive liquid medium. The electrodes are pulsed with high voltage direct current so as to create a spark that jumps over a gap between the two electrodes of each emitter at each pulse. The spark within the conductive medium creates an energy wave that propagates through the liquid medium causing the balloon to physically provide a force against the lesion or thrombus. The energy propagation includes the creation of micro-bubbles that also facilitate the physical force. Such intravascular lithotripsy (IVL) devices can be provided in different designs and sizes, wherein examples of IVL devices and systems can be found in the following patent applications: US Patent Application Pub. Nos. 2024/0156478, 2024/0180569, and 2024/0307081; International Publication Numbers WO 2023/015047, WO 2024/138212, and WO 2024/107470, the entire contents of which are hereby incorporated by reference. With this exchangeable handle design, physicians can minimize the number of steps to transition from piloting to treatment of the uncrossable lesion.

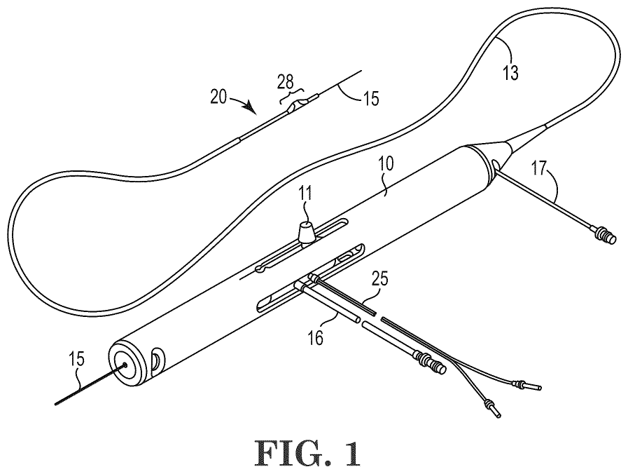

Referring now to the Figures, wherein the components are labeled with like numerals throughout the several Figures, and initially to FIG. 1, a schematic drawing is provided of a typical known rotational atherectomy device, wherein various embodiments of the present invention comprise a rotational atherectomy system including a number of the same features and elements described below. It is contemplated that one or more features, including configurations, placement, location, operational and functional characteristics, etc., of the various non-limiting exemplary embodiments of any and all abrading elements and systems are equally or substantially equally applicable for the piloting elements of the instant disclosure. One or more of such piloting elements may be provided individually and/or in combination with one or more abrading elements.

The device of FIG. 1 includes a handle portion 10, an elongated, flexible drive shaft 20 having an eccentric enlarged abrading head 28, and an elongated catheter 13 extending distally from the handle portion 10. The drive shaft 20 is constructed from helically coiled wire as is known in the art and the abrading head 28 is fixedly attached thereto. The catheter 13 has a lumen in which most of the length of the drive shaft 20 is disposed, except for the enlarged abrading head 28 and a short section distal to the enlarged abrading head 28. The drive shaft 20 also contains an inner lumen, permitting the drive shaft 20 to be advanced and rotated over a guide wire 15. A fluid supply line 17 may be provided for introducing a cooling and lubricating solution (typically saline or another biocompatible fluid) into the catheter 13.

The handle 10 desirably contains a rotational drive, such as a turbine driven by fluid pressure or an electric motor, for rotating the drive shaft 20 at high speeds. The handle 10 typically may be connected to a power source, such as compressed air delivered through a tube 16 or by an electrical cord from an AC or DC power source. One or more fiber optic cables 25 are illustrated and may also be provided for monitoring the speed of rotation of the rotational drive and drive shaft 20 (details regarding such handles and associated instrumentation are well known in the industry, and are described in U.S. Pat. No. 5,314,407 issued to Auth, for example, which is incorporated by reference herein in its entirety). The handle 10 also desirably includes a control knob 11 for advancing and retracting the rotational drive and drive shaft 20 with respect to the catheter 13 and the body of the handle 10.

The abrasive element 28 in FIG. 1 can be an eccentric solid crown that is attached to the drive shaft 20 near the distal end of the drive shaft 20. The term “eccentric” is used herein to denote that the center of mass of the abrasive element 28 is laterally displaced away from the rotational axis of the drive shaft 20. As the drive shaft rotates rapidly, the displaced center of mass of the abrasive element 28 causes the drive shaft to flex radially outward in the vicinity of the abrasive element 28 as it spins, so that the abrasive element 28 may abrade over a larger diameter (a working diameter) than its own rest diameter. Eccentric solid crowns are disclosed in detail in, for example, U.S. patent application Ser. No. 11/761,128 (U.S. Patent Publication No. US2008/0306498) to Thatcher et al., which is incorporated by reference herein in its entirety. It is noted, however, that a device that instead incorporates a concentric crown can be provided in accordance with embodiments described herein.

An electric motor may be included in the handle, or may instead include an air-fed or nitrogen-fed turbine, as is also known. In this respect, many or all of the other elements of the atherectomy device of FIG. 1 may be used with the present disclosed atherectomy device, including the catheter 13, the guide wire 15, the control knob 11 on the handle 10, the helically coiled drive shaft 20 and the eccentric solid abrasive element 28.

There are many combinations of features that may be included with the electrical device of the present invention. For instance, a device having relatively few features may be less expensive to produce than a relatively feature-laden device, and may be sold and marketed as such. Likewise, a device having many features may be sold and marketed as a high-performance device, which may command a higher price than the relatively feature-free device.

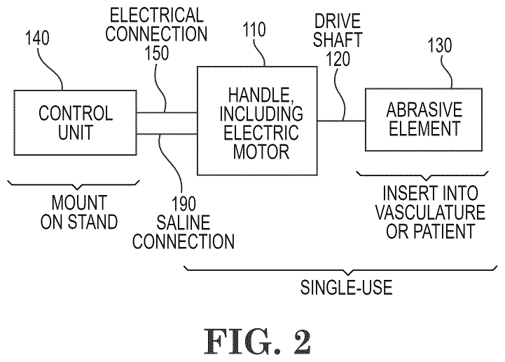

FIG. 2 is a block diagram of an atherectomy device having an electric motor and relatively few features, wherein a control unit 140 is a non-disposable portion of an atherectomy system that may be reused for multiple procedures. The control unit may be mounted on a stand, as noted in FIG. 2, or may function as a stand-alone device that may be placed on a countertop or the like. The device further includes a single-use or disposable portion that comprises a handle 110 with an electric motor that is connected to a rotatable drive shaft 120. An abrasive element 130 is attached to the drive shaft 120, wherein the abrasive element 130 is insertable into the vasculature of a patient.

The control unit 140 preferably has an electrical connection 150 with the handle 110. In many cases, the control unit 140 functions as a power supply for the motor in the handle 110, and the electrical connection 150 includes the two conductive elements required for current flow (or optionally three conductive elements, if a separate ground is used). Typically, the control unit 140 supplies a controllable and variable DC voltage to the handle 110, with the voltage varying in an open-loop fashion to control the rotational speed of the motor in the handle 110. Note that an AC voltage may also be used. For this schematically illustrated embodiment of the electrical connection 150, no communication is provided between the handle 110 and the control unit 140, as the control unit 140 simply powers the motor in the handle 110. Note that in other cases, the electrical connection 150 may be more sophisticated and may include one-way or two-way data communication between the control unit 140 and the handle 110, where such a case is described below.

The control unit 140 can also include a reusable saline pump. Such a pump directs saline at a predetermined rate from a bag or other suitable source through a saline connection 190 and into the handle 110. Suitable plumbing inside the handle 110 directs the saline into the catheter, where it fills the space surrounding the drive shaft and serves to lubricate and clean the system. The control unit 140 should preferably regulate the rate at which saline is pumped into the handle, and preferably also informs the operator of the status of the pump. These two functions are described below.

The saline pump preferably uses two pump rates, which are commonly designated as “low” and “high”. Typically, the low and high speeds are hard-coded in the firmware of the control unit 140. Alternatively, more than two discrete pump rates may be used, and/or a continuously varying pump rate may be used. Typically, the low pump rate is used to flush the system at the beginning of the procedure before the drive shaft begins its rapid rotation. The high pump rate is typically used during the procedure, when the drive shaft is rotating rapidly. In some cases, the pump rate is varied between high and low automatically, depending on the control unit power supply setting and/or the desired rotational speed of the motor in the handle. In some cases, at the beginning of each procedure, the user is instructed to turn the pump on at a low flow rate, to wait for a particular time, and to then turn the flow rate up to the higher rate.

The device may use a weight sensor to monitor the level of the saline. Such a weight sensor may be a spring-like device from which a saline bag is hung. If the hung weight of the bag and its contents drops below a predetermined threshold, a switch in the weight sensor can be triggered. The saline typically arrives in a standard-sized bag, such as 200 milliliters, although any bag size may also be used. A weight sensor may also be used on a platform-like device, on which the saline bag may be placed. If the weight of the bag drops below a predetermined level, then the pump is turned off, the motor is powered down (to prevent damage to the device and to the patient that might occur from running the device without saline), and the operator can be notified.

The operator can be notified of the pump system status through the control unit 140. In one embodiment of a notification system, the status can be provided by three differently-colored light emitting diodes (LEDs), such as can be provided on the control unit 140. A “green” light may indicate that the pump is operating normally and that the handle 10 is powered properly. There can be an internal circuit that monitors a power supply (preferably a 48-volt power supply) for the handle 10. A “yellow” light may indicate that something is not operating correctly (e.g., a door may be open or there may be a different correctable problem). A “red” light may indicate that the bag has run out of saline, for example. It will be understood that other suitable indication systems may be used as well.

In alternative designs, an electric motor is included within the control unit 140, rather than within the handle 110, and the electric connection 150 is replaced by a mechanical connection to transfer the rotation of the motor to the drive shaft.

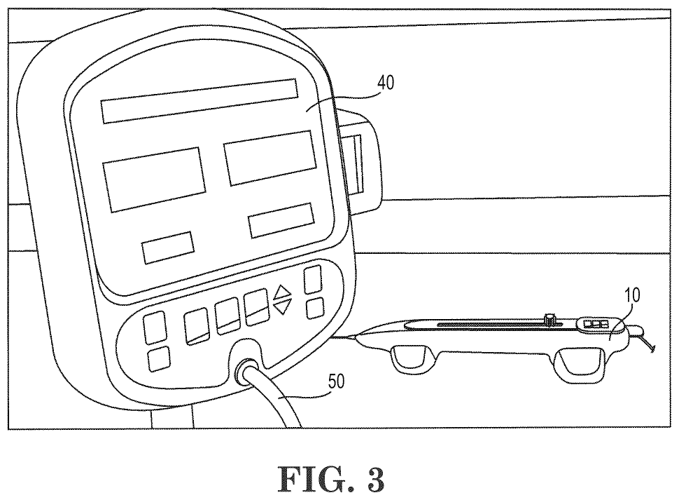

FIG. 3 is an illustration of an exemplary control unit 40 and handle 10. In this example, the electrical connection 50 extends from the front of the control unit 40 and connects to the handle 10, such as at a rear portion thereof. The catheter and drive shaft preferably extend from a front portion of the handle 10.

Many of the various device features, as discussed herein, are provided for convenience and are done so with respect to corresponding controls on the control unit 40. It will be understood that any suitable controls, with any suitable layout on the control unit 40, may be used for the described functions, and that the controls shown in the figures are merely examples. Such an exemplary control unit is described in detail in U.S. Patent Publication No. 2013/0253552 to Schoenle et al, the entire contents of which are hereby incorporated by reference.

A running speed can be indicated on the control unit 40 as the actual rotational velocity of the proximal end of the drive shaft, in units of 1,000 RPM (revolutions per minute), or kRPM. The running speed is typically updated several times per second, and in some cases may be displayed in relatively large LEDs that are readily visible to the practitioner. Rotational speeds of up to 200 kRPM are typical.

The rotational speed may be obtained from the electric motor itself. For instance, the motor may include one or more Hall effect sensors that produce an electrical signal each time the motor rotates past a particular point. The rotational speed is proportional to the rate of the signals, or, equivalently, is inversely proportional to the time intervals between the electrical signals. Alternatively, any suitable sensors and signals may be used. The control unit 40 can display a selected rotational speed. During operation, a control circuit (feedback loop) in the control unit 40 and/or the handle 10 can adjust the motor current and/or voltage to keep the actual running speed as close as possible to the selected speed.

An event time can be displayed as the elapsed time for a particular run of the atherectomy device. It is typical for the atherectomy device to be rated only for a particular time, beyond which use is not recommended. A device may be repeatedly turned off and on during the course of a full procedure. Such switching off and on is permissible as long as the total cumulative time during which the device is actually on does not exceed a particular value. Typically, the handle 10 includes electronics that store the cumulative on-time, although such data may alternatively be stored in the control unit 40.

If the total operational time hits the threshold value, the control unit may either shut down, or may emit a warning advising the practitioner that the on-time limit has been reached. In some cases, the limit can be overridden by the practitioner. In other cases, reaching the limit disables the motor so that the device can no longer be used.

Plural control buttons for speed selection can be provided and labeled “low”, “medium” and “high”, for example, with an indicator light on each that corresponds to the selected speed. In general, for a particular model of handle 10 that is connected to the control unit 40, there are preset speeds determined by the manufacturer. These speeds are automatically recognized by the control unit 40. Such recognition may take place by, for instance, storage of the preset speeds on the handle 10, storage of the preset speeds in a lookup table on the control unit 40, and/or lookups-as-needed of the preset speeds on a central database. If the practitioner desires more fine control of the speed than is offered by the default low/medium/high presets, increment buttons may adjust the selected speed upward or downward by a predetermined increment, such as 10 kRPM, although any suitable increment may be used.

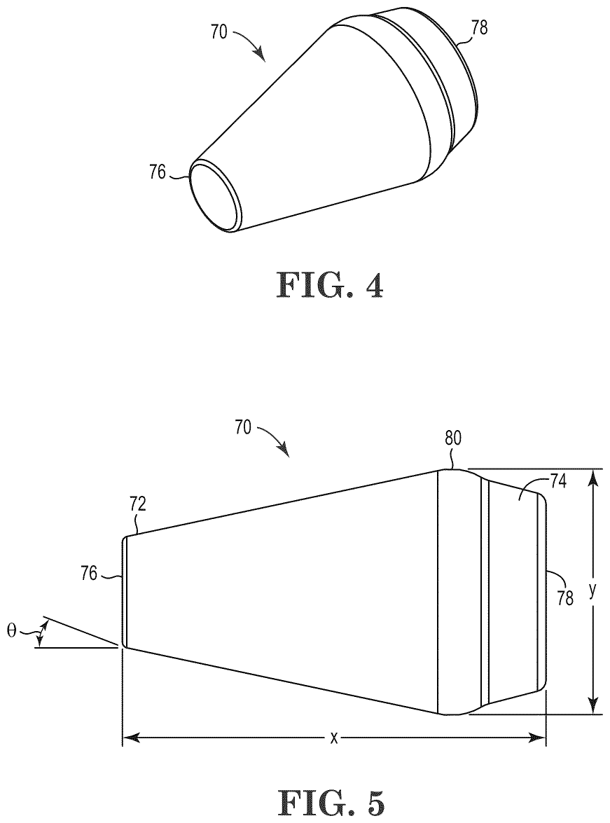

Referring now to FIGS. 4 and 5, an exemplary embodiment of a piloting head 70 is illustrated. Such a piloting head can be moved over a crossing wire to a lesion site and activated to pilot a hole through an otherwise uncrossable lesion. The device uses a forward-facing, diamond-coated crown attached to the end of a coiled driveshaft that provides bi-directional rotational atherectomy capabilities. When activated, the internal electric motor rotates the crown and driveshaft within the body, allowing the crown's diamond coating to engage and remove the calcified lesion material. By expanding the inner lumen of the lesion, the desired space is created within the lesion to deploy subsequent therapies. Although diamond coatings are discussed herein, it is contemplated that other abrasive coatings in addition to or instead of diamond coatings can be used for the tip.

The geometry of the piloting head 70 includes an exaggerated taper along a front face 72 that transitions to a radiused crest or shoulder and shorter taper at a rear face 74 before terminating. A tapering angle (θ) of the front face 72 provides successful engagement into relatively small openings and is primarily driven by the proportion of the overall length (x) of the front face 72 that the taper encompasses.

The following Table 1 shows bench data gathered from an evaluation of the time to move piloting heads having different configurations through a simulated lesion. As shown, the time to cross the simulated lesion was substantially longer once the taper angle (θ) exceeded 20°.

TABLE 1

| Tip Length (x) | Taper Angle (θ) | Time to Cross @ 80 krpm (sec) |
|---|---|---|
| .095″ | 9.0° | 15 |
| .085″ | 10.3° | 12 |
| .075″ | 12.0° | 15 |
| .050″ | 23.5° | 95 |

A radiused crest or shoulder (e.g., 0.01″) occurs at the maximum outer diameter (y) of the piloting head, providing a crossing profile around 1 mm. Other crossing profile diameters may also be utilized. The radiused nature of the crest or shoulder helps promote bidirectional engagement and sanding when working through a lesion by always providing a tapering edge to engage with the lesion regardless of which direction the piloting head is moving. The radiused crest also provides area for a diamond coating to adhere to the backside of the crown. The diamond coating on this area is important in providing the bidirectional sanding capability. The rear taper provides a zone for weld attachment of the piloting head to the driveshaft that promotes a smooth transition from one component to the other, thereby avoiding the creation of any unintended traumatic surfaces.

With continued reference to FIGS. 4 and 5, piloting head 70 includes a distal end application 76 having a diameter that is smaller than the diameter at its proximal end 78. As shown, the diameter increases at a constant taper angle (θ) until reaching a crest or shoulder area 80. The crest or shoulder area 80 generally includes a curved surface that then tapers down toward the proximal end 78 at a constant angle. It is understood, however, that the tapered angles of the various portions of the piloting head can be different from this illustrated exemplary embodiment, and may not be constant in the areas adjacent to the distal end 76 and proximal end 78. The piloting head 70 may be generally symmetrical about a central axis, as shown, or can be asymmetrical about the central axis.

The piloting head 70 further includes an outer surface, and an inner surface that defines a lumen. The outer surface of the piloting head 70 is attached to the outer surface of a drive shaft and the inner surface of the piloting head 70 advances and rotates over a guide wire. In an embodiment, the piloting head 70 may be attached to a distal end of the drive shaft, and the lumen defined by the inner surface allows the piloting head to be advanced and rotated over a guide wire.

FIG. 10 illustrates several exemplary piloting heads 370 having different configurations (i.e., different lengths, diameters, and angled surfaces), but that include the various features described herein (i.e., angled surfaces, shoulder/crest area, etc.). Piloting heads 370 may have an abrasive coating on some or all of their respective outer surfaces, wherein the placement and selection of abrasive will be selected to match the particular environment in which it will be used. As discussed herein, the abrasive coating may be a diamond coating having a desired grit size.

When used in conjunction with an exchangeable handle platform, larger diameter configurations of the piloting head can also be used. After utilizing the previously described approximately 1 mm diameter crown size, the exchangeable cartridge can be replaced with a separate cartridge attached with a larger diameter crown. These larger diameter crowns can be approximately 1.25 mm or 1.50 mm, for example, or another size, in line with available rotational atherectomy systems. The larger crown sizes can then be utilized after the initial piloting crown to further remove calcified material at the treatment site via atherectomy. Cartridges with larger rotational crowns can therefore facilitate easier deployment of subsequent therapies for physicians wanting to perform further rotational atherectomy on a lesion site, as cartridges can be attached to the same base handle.

Due to the difficulty crossing CTOs with a guide wire, it is desirable for the rotational atherectomy device to operate compatibly with an existing guide wire. Depending on the operator's preference, these wires can include a variety of coatings (e.g., hydrophilic, hydrophobic, silicone) and/or polymer jackets that help to maximize the deliverability and crossability of the wire. Therefore, the rotational atherectomy device minimizes disruption of these coatings to promote safe outcomes of the procedure and continued use of the wire with subsequent therapy devices.

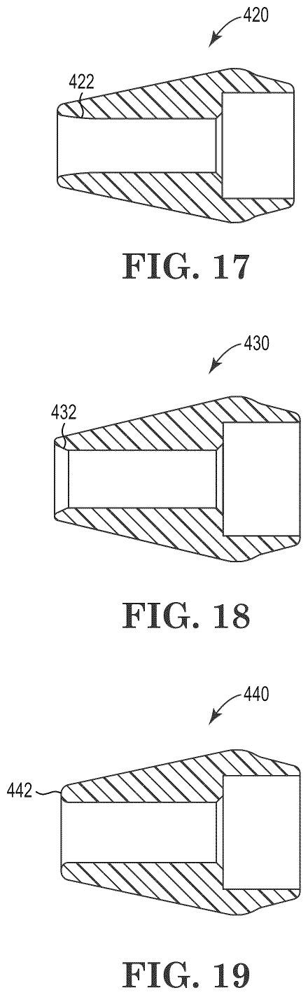

The piloting heads described herein can aid in minimizing disruption of coatings on the existing guide wire by reducing the interaction between sharp edges present on the head with the guide wire coatings. The internal surface of the piloting head can therefore incorporate various implementations of a smoothed inner flare to remove sharp edges at the distal end of the piloting head, which may otherwise cut away at fragile guide wire coatings. To accomplish this, FIG. 17 illustrates a piloting head 420 that includes an inner flare 422 that is a generally smooth radius from the inner diameter of the piloting head to the front edge, wherein the angle and length of the flare can be different from that illustrated, FIG. 18 illustrates a piloting head 430 that includes a chamfered edge 432 from the inner diameter of the piloting head to its front edge, or other edge-smoothing configurations can be utilized. FIG. 19 illustrates another embodiment of a piloting head 440 having a crown with an exaggerated radius 442 on the front edge of the head that can achieve the same purpose. Implementing a radius on the front edge of the head reduces some portion of the overall length of the head in order to smooth the surface of the front edge.

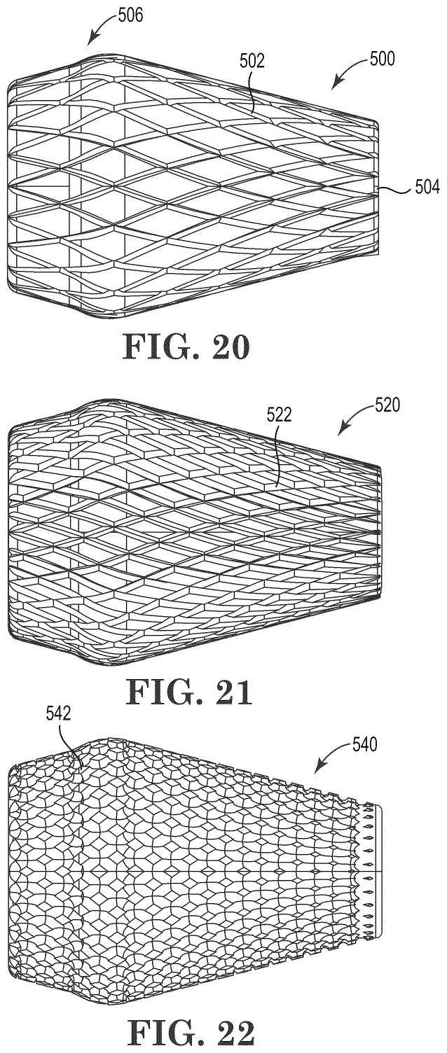

As discussed above, atherectomy devices operate via a motor spinning a flexible driveshaft at a high rate of speed to allow a distal abrasive element (crown) to perform work on a target lesion. The atherectomy crown can be coated with an abrasive coating, such as diamond particles, to allow the device to effectively remove hardened plaques during use, as is also discussed above. An alternative to this use of an abrasive coating is to laser etch an abrasive surface or pattern onto the desired areas of the crown, wherein examples of such laser etchings are illustrated in FIGS. 20-22, described below.

FIG. 20 illustrates an atherectomy crown 500 with a cut away helical pattern on its external surface. This pattern provides channels 502 along the external surface of the crown 500 with sharp edges, which act as abrasive elements for cutting away hardened plaque. The helical shape of the pattern promotes the advancement of the crown 500 into the lesion during use. As the pattern extends from the front distal edge 504 of the crown 500, the angle between the channel walls increases, with a smaller angle at the front edge 504 and a larger angle at the widest section 506 of the crown. Changing this angle also changes the cutting ability of different portions of the crown 500. In the front of the crown 500 where it is most aggressively engaging with the lesion, a smaller channel angle can be used to provide additional cutting ability. In the rear area of the crown, however, a larger angle can be used to limit the cutting ability of the crown in this section. This variable cutting ability will also limit the cutting ability of the crown in sections most likely to interact with the vessel walls. The chosen pattern could be laser cut into the surface of the crown, and programmed to accurately cover specific areas of the crown's geometry. The cut pattern can also extend up to, but preferably not over, the front face of the crown to assist in initial engagement with the lesion.

FIG. 21 illustrates another atherectomy crown 520 with a cut away helical pattern on its external surface. This pattern is similar to that of the crown 500 shown in FIG. 20, but crown 520 includes more channels 522. Adding more abrasive elements to the crown 520 can result in more effective engagement and treatment with the target lesion.

FIG. 22 illustrates another atherectomy crown 540 with individual nodules 542 extending from the external surface of the crown 540 that can each act as an individual cutting tool. A helical pattern is again added to drive the crown 540 into the lesion during use. These nodules 542 can be precisely located on the external surface of the crown to start and end at specific locations, so as not to cover the front face of the crown, and also to provide a clear weld attachment site for the device's driveshaft.

Other embodiments of this invention can include atherectomy crowns having a varying pitch helix, cutting feature profile changes (i.e., round channels transitioning to square channels), straight cutting features either axially or radially or a combination thereof, a separate feature for cutter geometry (i.e. a tip cutting feature separate from the OD cutting feature), an abrasive texture pattern rather than a cutting pattern on the exterior surface, or any combination thereof.

With any of the atherectomy crowns described herein, it is desirable to prevent interactions between the coating material of the crown (e.g., diamond coating) and the guide wire. One method of preventing such an interaction is to mask the crown during diamond coating to prevent diamond particles and coating solution from adhering to the front edge of the crown and its inner diameter. Such masking can be achieved through the use of custom designed spacers that fit around and cover the front geometry of the crown. When positioned in this way, the spacers seal against the front face of the crown, thereby preventing diamonds from migrating to areas that may cause interaction with the guide wire during use.

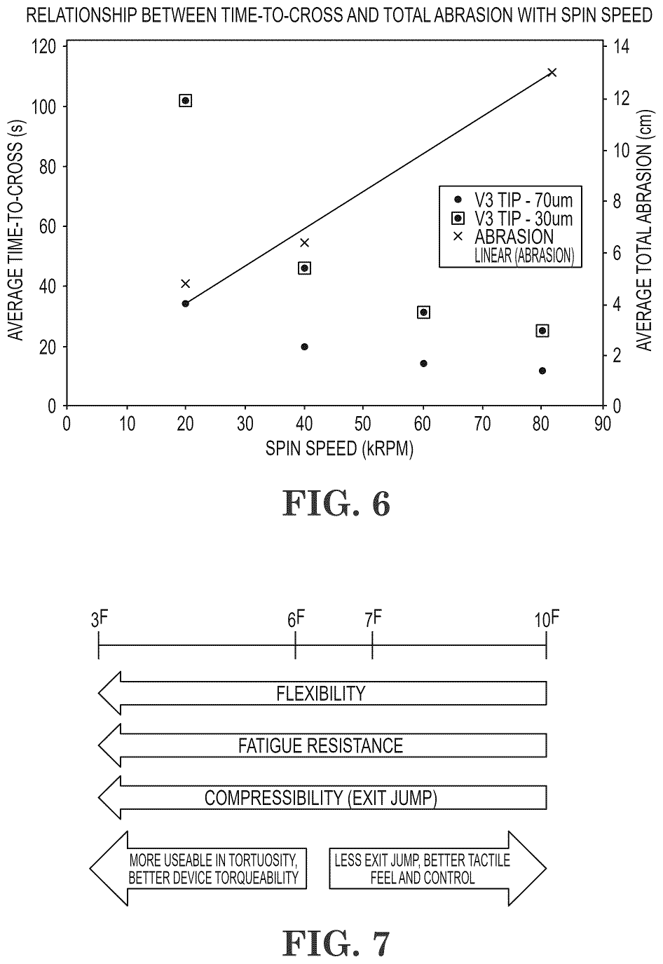

Referring now to FIG. 6, a graph is shown that compares the time to cross a calcified lesion and the amount of abraded coating from the guide wire with device spin speed. The spin speed of the devices described herein has been shown to impact the time to pilot across a calcified lesion and the amount of coating abraded from the guide wire. As the piloting head spins, a metal driveshaft coil interacts with a coating on the guide wire, potentially removing portions of the coating and exposing the bare metal core of the guide wire. This type of wire coating abrasion potentially increases the amount of particulate generated from operating the device and also potentially increases the amount of force required for a physician to deploy subsequent devices over the same guide wire.

Bench testing has shown that reducing the spin speed of the device linearly decreases the total amount of coating abrasion found on the guide wire after using the device and non-linearly increases the amount of time to cross the lesion. Additionally, increasing the diamond grit size on the device's distal tip has shown a non-linear decrease in the time to cross calcified lesions across all spin speeds. Therefore, to balance the minimization of coating abrasion with a faster time to cross, the piloting heads described herein can be used with a device that operates at a single speed near 40 kRPM, for example.

An exemplary driveshaft to which a piloting head of the type described herein is attached includes a wound coil made up of multiple stainless-steel wires (filars). The construction, based on the number of wires used and the size of the wires, will dictate how the coil will perform. FIG. 7 is a diagram that shows driveshaft variables with respect to the number of driveshaft filars used. In particular, this diagram illustrates performance impact based on the number of filars used and how that changes the flexibility, fatigue resistance, and compressibility of the coil. For instance, a driveshaft that has only 3 filars or wires will have greater flexibility, greater fatigue resistance, and greater compressibility as compared to a driveshaft that has 10 filars or wires.

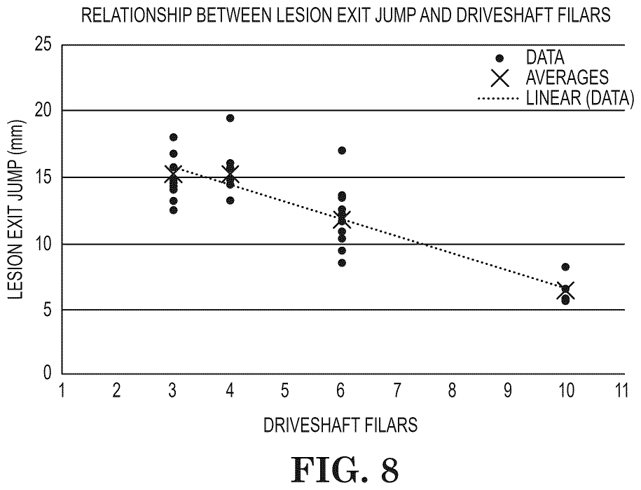

In operation, the driveshaft used will typically be designed to provide a balance between flexibility, control, and torqueability and tactile feel. As shown in the graph of FIG. 8, an inverse relationship has been found between the number of driveshaft filars and driveshaft compressibility. Additionally, operator feedback from bench testing indicates a direct relationship between the number of driveshaft filars and the amount of tactile feedback experienced by the device operator. In some cases, a balance of these performance variables can be found in a 6 filar coil.

The driveshaft generally includes an inner lumen for a guide wire to pass through during use. Most guide wires intended to be used with a device that includes the piloting heads described herein have a polymer jacket or coating along its exterior to aid in lubricity. When the driveshaft is spun this polymer can become disrupted and removed, potentially impacting the ability to utilize the wire for subsequent therapy. Thus, it is advantageous to spin the device over these coated wires, and several design alternatives are possible to use with the present invention, a number of which are described below.

In one design alternative, a polymer ID liner is provided, which involves the placement of a thin polymer tube within the inner diameter of the driveshaft to act as a barrier between the metal filars and guide wire coating. Since the wire cannot interact directly with the driveshaft in this case, disruption or removal of the wire coating is greatly reduced or eliminated. The liner can extend the full length of the driveshaft's inner lumen or be shortened to protect certain areas where extra guide wire protection is needed. The length of the liner can be optimized to balance the stiffness of the driveshaft-liner assembly with desired coverage of the guide wire. The liner may be secured in place at its proximal end using an adhesive or other securement method to prevent the liner from shifting its position during use of the device. Potential material choices for the liner include polyimide or PEEK.

In another design alternative, a coated or alternate material coil is provided, which involves either applying a polymer to the surface of the coil filars or utilizing a different material altogether to obtain a more positive interaction between the guide wire and coil. The coil could be coated through a dip process after winding or have it applied to the individual filars prior to winding. This results in a polymer-to-polymer interaction that can be more favorable to maintaining the wire coating.

Similarly, winding the coil with an alternate material, like Nitinol, can provide a more favorable interaction. Such an alternate material can be formed into coils to create the driveshaft, and then stabilized to maintain its coiled shape through additional processing, such as heat setting. The temperature and timing of the processing used to set the alternate material may vary to produce the desired performance characteristics for the driveshaft.

In another design alternative, a polymer driveshaft is provided, which includes the construction of a true polymer driveshaft that leverages approaches utilized in catheter constructions. The driveshaft includes inner and outer polymer layers sandwiching a coil or braid that provides added strength and reinforcement.

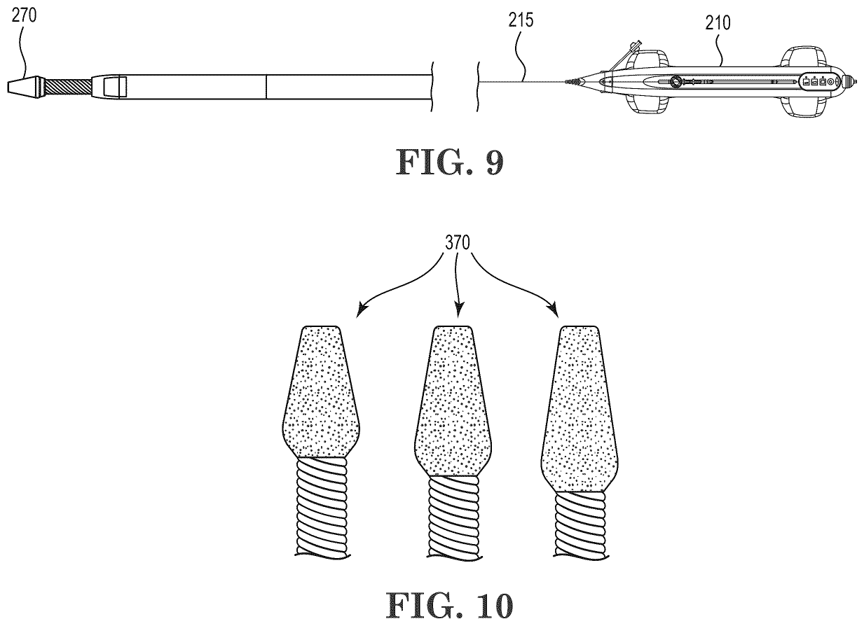

FIG. 9 is an illustration of a handle 210 in accordance with the present invention. An electrical connection from a control unit extends from the handle 210 at one end, while a catheter and drive shaft extend from the handle 210 at its other end. The handle 210 can include a control knob, as discussed above, for longitudinally translating a drive shaft with respect to both a guide wire and a catheter, which remain stationary. In accordance with the present invention, the control knob is optional in that the translational movement that can be imparted to the drive shaft and thus a piloting head 270 can instead be provided by a linear drive device, for example. Translational movement of the piloting head 270 is preferably permitted over a desired range by the movement of the linear drive device. Preferably, the piloting head 28 is movable along a travel range of distances. Back and forth movements of the piloting head 270 can be used during the process of creating a pilot passage through a blockage.

The handle 210 may include a duplicate set of speed selection buttons, which can repeat the functionality of the corresponding buttons on the control unit. Having speed selection buttons on the handle 210 itself can be a convenience for the practitioner. The handle 210 may further include an extending electrical connection, which can be a 14-foot-long cable, for example, although other suitable lengths may be used. The drive shaft extending from the handle 210 may be a helically-wound coil of wire, although any suitable mechanism for delivering torque from the electric motor to the piloting head 270 may be used as a drive shaft. For instance, an alternative drive shaft may be a solid or slotted tube of plastic or metal. The piloting head 270 is shown at the end of a drive shaft, with an abrasive material coated on the exterior of the piloting head.

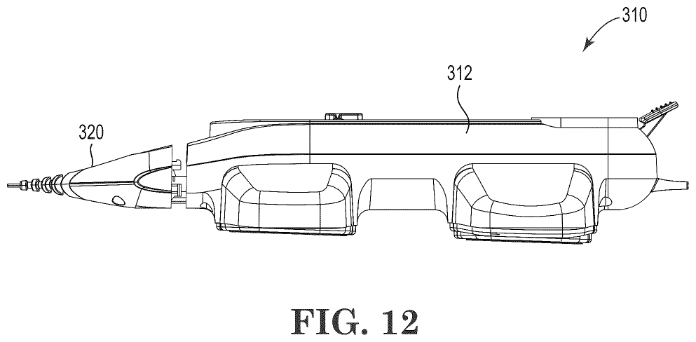

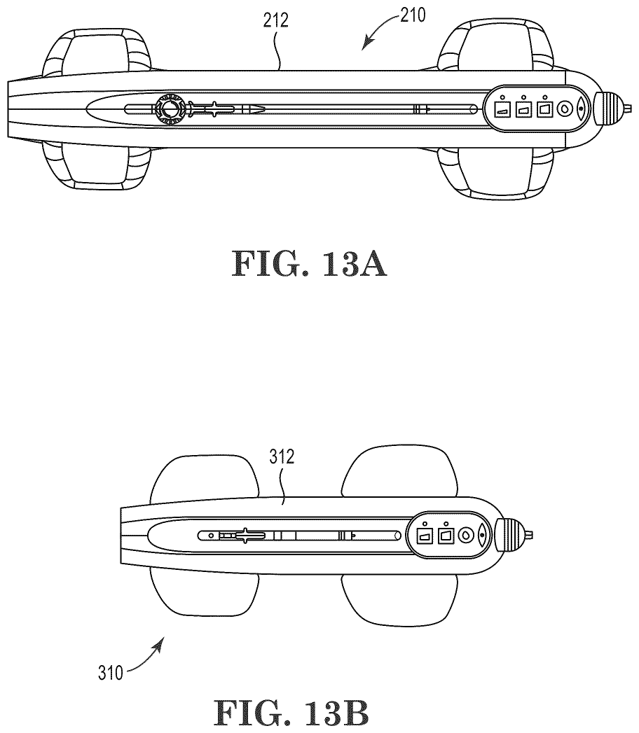

FIGS. 12, 13B, 14, and 15 are illustrations of a handle 310 and certain components thereof in accordance with an embodiment of the present invention, wherein FIG. 13A illustrates the handle 210 discussed above relative to FIG. 9 for comparison purposes. As is shown in FIGS. 13A and 13B, handle 210 includes a body member 212 that is at least somewhat longer than a body member 312 of handle 310. The shorter handle 310 provides a more compact design that can better accommodate certain lengths of guide wires (e.g., 190 cm guide wires), which can be desirable in some coronary vascular procedures. That is, a shorter travel distance for the device can be useful for performing certain atherectomy procedures in the coronary vasculature.

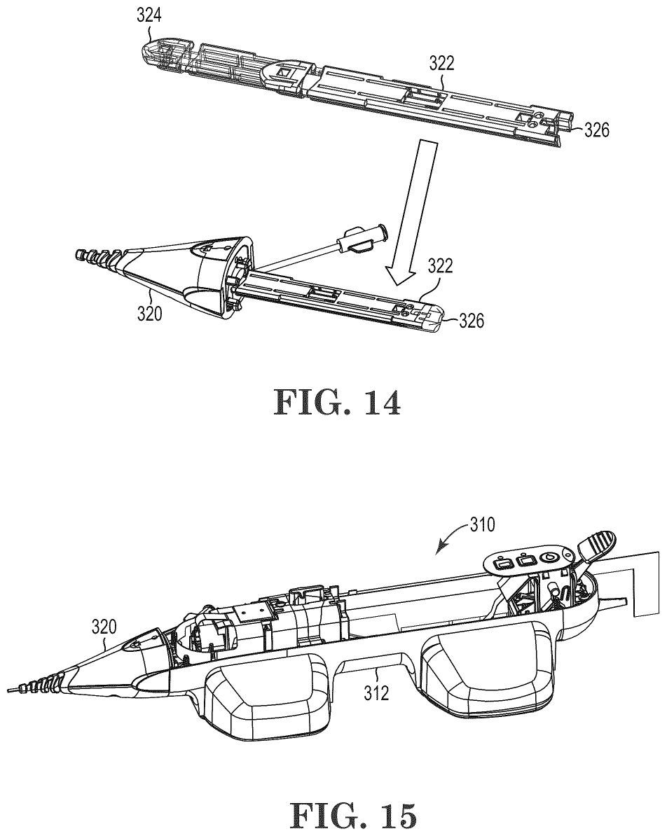

FIG. 14 illustrates an exemplary connector member 322 that is used to attach an exchangeable cartridge 320 to the handle 310. In particular, one end 324 of connector member 322 connects to cartridge 320, such as via insertion into an opening in cartridge 320, while the opposite end 326 of connector is configured to connect with the handle 310 via insertion into an opening in cartridge 320. FIG. 15 illustrates an exemplary configuration of internal handle components within the shorter body member 312.

With regard to handle 310, an electrical connection from a control unit will extend from the handle 310 at one end, while a catheter and drive shaft extend from the handle 310 at its other end. The handle 310 can include a control knob, as discussed above, for longitudinally translating a drive shaft with respect to both a guide wire and a catheter, which remain stationary. In accordance with the present invention, the control knob is optional in that the translational movement that can be imparted to the drive shaft and thus a piloting head can instead be provided by a linear drive device, for example. Translational movement of the piloting head is preferably permitted over a desired range by the movement of the linear drive device. Preferably, the piloting head is movable along a travel range of distances. Back and forth movements of the piloting head can be used during the process of creating a pilot passage through a blockage.

The handle 310 may include a duplicate set of speed selection buttons, which can repeat the functionality of the corresponding buttons on the control unit. Having speed selection buttons on the handle 310 itself can be a convenience for the practitioner. The handle 310 may further include an extending electrical connection, which can be a 14-foot-long cable, for example, although other suitable lengths may be used. The drive shaft extending from the handle 310 may be a helically-wound coil of wire, although any suitable mechanism for delivering torque from the electric motor to the piloting head may be used as a drive shaft. For instance, an alternative drive shaft may be a solid or slotted tube of plastic or metal.

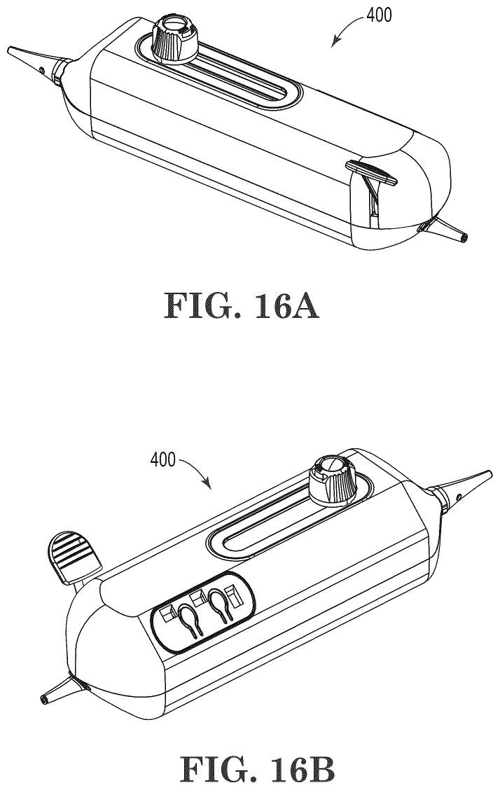

A dedicated handle platform that does not include exchangeable functionality can also be utilized, as is illustrated with the rotational atherectomy device handle 400 of FIGS. 16A and 16B. This standalone design incorporates the ability to remove a deployed guide wire and backload a new guide wire through the handle rear strain relief. The handle components can be simplified to remove any additional mechanisms required for the subsequent use of orbital atherectomy or higher speed rotational atherectomy, thereby reducing the amount of required internal space. The reduction in components and design complexity produces a lower cost handle platform with a shorter handle length profile. The shorter handle length profile allows physicians the ability to deploy their guide wire further into the coronary anatomy while still maintaining control of the guide wire when using a trapping balloon deployment technique.

A method for providing a pilot hole through a lesion in a blood vessel generally includes the following steps: providing a guide wire with a maximum diameter that is less than the inner diameter of the blood vessel; advancing the guide wire across the stenosis/lesion; providing a flexible elongated, rotatable drive shaft advanceable over a guide wire, the guide wire having a maximum diameter less than the diameter of the artery; the drive shaft having a rotational axis and a piloting head fixedly attached to the drive shaft; advancing the piloting head into the artery to a position proximal to the lesion; creating a piloting hole by rotating the drive shaft at a sufficient rotational speed; and advancing the crown to engage the lesion using the translational and back and forth movements described herein.

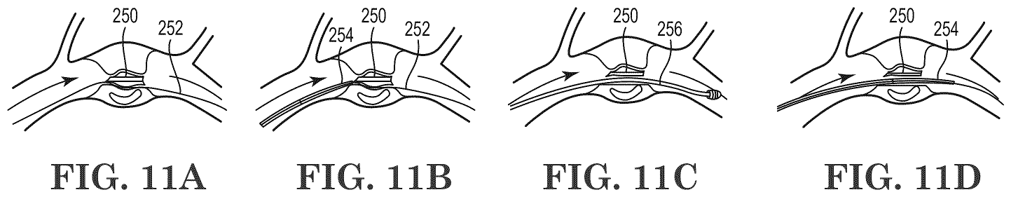

FIGS. 11A-11D schematically illustrate the above-described method in four sequential steps of using a rotational atherectomy device in a vessel to create a pilot hole in a lesion before being able to use one or more devices to further treat the vessel. In particular, FIG. 11A illustrates a lesion 250 that significantly blocks a vessel opening, wherein a guide wire 252 has passed by the lesion. However, a treatment device 254 cannot move over the guide wire 252 to cross the lesion 250, as illustrated in FIG. 11B, as there is no space for it to pass. FIG. 11C illustrates a rotational atherectomy device 256 of the invention as it has removed at least a portion of the lesion 250 so that treatment device 254 can then pass by the lesion 250, as is illustrated in FIG. 11D.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A device for creating a pilot hole through lesions in body passageways, the device comprising:
an elongated flexible drive shaft advanceable over a guide wire, the drive shaft comprising a proximal end and a distal end; and
a piloting head fixedly attached to at least a portion of the distal end of the driveshaft, wherein the piloting head comprises a distal end having a first diameter, a proximal end having a second diameter that is larger than the first diameter, and an intermediate shoulder area comprising a third diameter that is larger than the first and second diameters,
wherein the intermediate shoulder area comprises a radiused crest having a backside area comprising an abrasive coating; and
wherein the diameter of the piloting head tapers down at a generally constant angle from the intermediate shoulder area to the proximal end of the piloting head.

2. The device of claim 1 in combination with a handle from which the drive shaft extends, wherein the handle and extending drive shaft are removable and replaceable with a replacement handle from which a replacement drive shaft extends.

3. The device of claim 1, wherein the drive shaft and piloting head comprise a removable cartridge that is replaceable with an atherectomy cartridge.

4. The device of claim 1, wherein the intermediate shoulder area is located at a maximum outer diameter of the piloting head.

5. The device of claim 1, wherein the radiused crest comprises a diamond coating.

6. The device of claim 1, wherein the piloting head comprises a radiused inner flare adjacent to the distal end.

7. The device of claim 1, wherein the piloting head comprises a chamfered inner flare adjacent to the distal end.

8. The device of claim 1, wherein the piloting head comprises an exterior surface, and wherein at least a portion of the exterior surface comprises a cutter pattern.

9. The device of claim 8, wherein the cutter pattern comprises at least one of a varying pitch of cutter grooves or channels and a varying angle of cutter grooves or channels.

10. The device of claim 8, wherein the cutter pattern comprises at least one of a varying geometry or shape of cutter grooves or channels.

11. The device of claim 8, wherein the cutter pattern comprises a varying number of cutter grooves or channels.

12. The device of claim 1, wherein the portion of the driveshaft proximal to the piloting head is free of any abrasive coating for imparting an orbital motion in a proximal portion of the drive shaft.

13. The device of claim 1, wherein the diameter of the piloting head tapers up at a generally constant tapering angle from the distal end to the intermediate shoulder area of the piloting head.

14. The device of claim 13, wherein the tapering angle is less than 20 degrees.
